(19) United States
(12) Patent Application Publication
(10) Pub. No.: US 2026/0083380 A1
(43) Pub. Date: Mar. 26, 2026

(54) SYSTEM AND METHOD FOR ELECTRODE-BASED ELECTROCARDIOGRAM MONITORING AND ANALYSIS USING MOBILE DEVICES

(71) Applicant: E-MBRYONIC LLC, Yardley, PA (US)

(72) Inventor: Michael Montgomery, Yardley, PA (US)

(21) Appl. No.: 19/339,914

(22) Filed: Sep. 25, 2025

Related U.S. Application Data
(60) Provisional application No. 63/699,147, filed on Sep. 25, 2024.

(51) Int. Cl. A61B 5/333; A61B 5/00; A61B 5/024; A61B 5/28; A61B 5/332; A61B 5/339; A61B 5/364

(52) U.S. Cl. CPC A61B 5/333; A61B 5/0006; A61B 5/0024; A61B 5/02405; A61B 5/28; A61B 5/332; A61B 5/339; A61B 5/364; A61B 5/7203; A61B 5/746; A61B 5/7475; A61B 2560/0487; A61B 2562/08

(57) ABSTRACT

Systems, methods, and devices are disclosed for wireless electrocardiogram (EKG) monitoring. An example method may detect wireless electrodes placed on a patient's body, each with a unique identifier. The example method may also display optimal electrode placement locations on the device's screen. Additionally, the example method may verify the placement of the wireless electrodes using the mobile device. The example method may also transmit voltage measurements from the electrodes to the mobile device. Additionally, the example method may integrate the voltage measurements into an EKG reading using the mobile device.

### CROSS-REFERENCE TO RELATED APPLICATION

This application claims priority to U.S. Provisional Ser. No. 63/699,147 , filed on Sep. 25, 2024, which is herein expressly incorporated by reference in its entirety for all purposes.

### TECHNICAL FIELD

The present disclosure relates generally to wireless electrocardiogram (EKG) technology and, more specifically, to wireless electrode-based EKG monitoring and analysis using mobile devices.

### BACKGROUND

Electrocardiograms (EKGs) are diagnostic tools for assessing heart health. Traditionally, they involve cumbersome wired systems that require skilled technicians to place numerous electrodes correctly on the patient. This process can be time-consuming and complex, limiting patient mobility and comfort, and making it challenging to perform EKGs in various environments, such as during physical activity or in remote locations.

Current advancements have introduced some wireless EKG solutions. However, they often fail to provide a complete 12-lead EKG or require proprietary hardware and software. These solutions can be costly, may not integrate seamlessly with existing medical record systems or consumer electronics, and often rely on limited lead configurations that reduce diagnostic accuracy.

Furthermore, the lack of standardization in wireless communication protocols between devices complicates the seamless transmission of EKG data to electronic health records (EHRs) and other medical databases. These interoperability issues hinder efficient data sharing and continuity of care, which are crucial for effective patient management.

There is a growing need for a more efficient, cost-effective, and user-friendly EKG solution that leverages modern wireless technology and the ubiquity of mobile devices. Such a system would simplify obtaining accurate EKG readings, enhance patient comfort and mobility, and facilitate broader adoption in various medical, sports, and military applications.

### SUMMARY

The following presents a simplified summary of one or more aspects to provide a basic understanding of such aspects. This summary is not an extensive overview of all contemplated aspects and is intended to neither identify key or critical elements of all aspects nor delineate the scope of any or all aspects. Its sole purpose is to present some concepts of one or more aspects in a simplified form as a prelude to the more detailed description that is presented later.

Devices, systems, and methods for wireless EKG monitoring may involve using a mobile device to detect and verify the placement of wireless electrodes with unique identifiers on a patient's body. The system may display optimal electrode placement, transmit voltage measurements from the electrodes to the mobile device, and integrate these measurements into an electrocardiogram (EKG) reading. The method ensures accurate data collection and real-time monitoring, enhancing the efficiency and convenience of traditional EKG procedures.

Systems, methods, and devices are disclosed for wireless EKG monitoring. An example method may detect wireless electrodes placed on a patient's body, each with a unique identifier. The example method may also display optimal electrode placement locations on the device's screen. Additionally, the example method may verify the placement of the wireless electrodes using the mobile device. The example method may also transmit voltage measurements from the electrodes to the mobile device. Additionally, the example method may integrate the voltage measurements into an electrocardiogram (EKG) reading using the mobile device.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a diagram illustrating the hardware components of a device.

FIGS. 2A-2C illustrate a flowchart outlining the method for wireless EKG monitoring.

FIGS. 3A-3C is a data flow diagram illustrating imaging, electrode placement, and EKG processing within the EZG system.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of configurations. It is not intended to represent the only configurations in which the concepts described herein may be practiced. The detailed description includes specific details to provide a thorough understanding of various concepts. However, it will be apparent to those skilled in the art that these concepts may be practiced without these specific details. In some instances, well-known structures and components are shown in block diagram form to avoid obscuring such concepts.

Embodiments of the systems, methods, and devices described herein may have one or more of the following capabilities. For example, one embodiment of the systems, methods, and devices described herein may include an advanced electrocardiogram (EKG) system utilizing wireless electrodes, mobile devices, and centralized hubs for efficient and effective heart monitoring and analysis. The system may replace traditional wired EKG setups with a more user-friendly, cost-effective, and versatile solution.

User scenarios demonstrate the system's ability to enhance patient comfort by eliminating restrictive wires and allowing for more natural movement during the monitoring process. In clinical settings, this can lead to more accurate readings and improved patient experiences. The system's portability also allows for use in remote or field environments, making it suitable for military applications, sports applications, emergency medical applications, or any other application where portability may be an asset. (It will also be understood that a portable system may generally still be used in a fixed application.)

The integration with mobile devices ensures that the system leverages the latest in processing power and display technology, offering a user-friendly interface and real-time processing capabilities. This makes it easier for healthcare providers to quickly interpret and act on EKG data, improving the overall efficiency of the diagnostic process.

### System Overview

Some embodiments of the systems described herein may include a device 100, which integrates various components for performing wireless EKG monitoring. The device 100 may be configured to interact with an iPad, iPhone, VisionPro, Android based devices, other manufacturers' mobile devices, or a similar mobile device capable of processing and displaying EKG data.

The application (e.g., App) designed for this system includes intuitive user interfaces that guide users through the electrode placement process, provide real-time feedback on signal quality, and offer detailed visualizations of the EKG data. The App's real-time processing capabilities ensure immediate analysis and display of the heart's electrical activity, allowing for quick and informed decision-making by healthcare professionals.

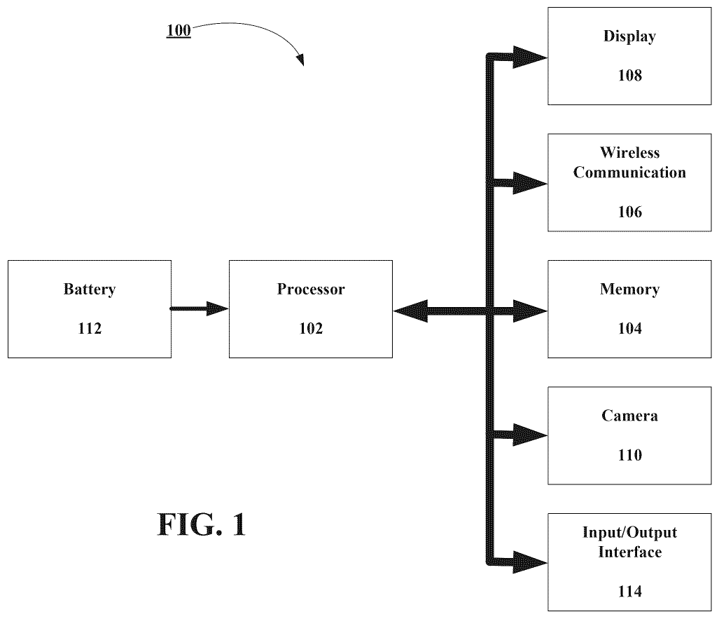

FIG. 1 is a diagram illustrating the hardware components of a device 100. Device 100 may include one or more processors 102. One or more processors 102 may handle the data processing tasks required for analyzing the signals received from the wireless electrodes. Device 100 may include memory 104. Memory 104 may store the software application (App) that manages the data collection, analysis, and display.

Device 100 may include a wireless communication module 106. Wireless communication module 106 may facilitate communication between the wireless electrodes and the device using standards such as Thread, Bluetooth, Zigbee, Z-Wave, LoRa, Wi-Fi, or other current or future wireless communication protocols suitable for medical or consumer electronics applications. Wireless communication module 106 may include display 108. In other embodiments, the display 108 may be a separate device, Display 108 may show the real-time EKG data and provide visual guidance for electrode placement.

Device 100 may include one or more cameras 110. For example, device 100 may include a front-facing camera and rear-facing camera, which may be used to recognize the thoracic anatomy and assist in proper electrode placement. Device 100 may include battery 112. For example, the device 100 may be powered by an internal battery (battery 112), ensuring portability and ease of use in various environments. Device 100 may include Input/Output Interfaces 114. These interfaces may allow the device to connect to other systems, such as EHRs, printers, and hubs, for further data processing and storage.

In an example embodiment, one or more of the functions of device 100 may be implemented on a mobile electronic device such as an iPad, iPhone, VisionPro, or another mobile device capable of processing and displaying EKG data.

In some embodiments, the device may comprise or interact with emerging wearable visualization devices, such as augmented-reality (AR) or mixed-reality (MR) glasses. Examples include Apple VisionPro, Meta Ray-Ban devices, or other current or future wearable display technologies. These devices may provide a hands-free, heads-up interface for displaying electrode placement guidance, real-time electrocardiogram (EKG) waveforms, and system feedback. Such wearable devices may supplement or substitute traditional handheld mobile devices to enhance usability in clinical, athletic, or field environments.

### Functional Description

The system may operate using wireless electrodes placed on the patient's body. These electrodes may adhere to the skin using adhesive patches and may measure voltage changes from the heart's electrical impulses. Each electrode may have a unique identifier signal, enabling the system to recognize its position and function.

### Electrode Placement and Detection

The application, running on device 100, may use camera 110 to scan the patient's thoracic anatomy. The application may display the optimal locations for electrode placement on the device's display 108. Once the electrodes are placed, the application may verify their positions using the camera and provide feedback if any electrodes are incorrectly placed or missing.

In some embodiments, electrodes may be identified not only by a unique identifier but also, or alternatively, by their initial placement positions on the patient's body. For example, the system may analyze the relative positions of electrodes using imaging data, computer vision, or artificial intelligence (AI) algorithms, and automatically assign anatomical labels such as right arm (RA), left arm (LA), right leg (RL), left leg (LL), or chest positions (V1-V6). This positional inference may supplement or substitute for unique identifier signals, providing redundancy and flexibility in cases where identifiers are unavailable, duplicated, or not required.

### Data Collection and Integration

Wireless communication module 106 may receive signals from the electrodes. These signals may be transmitted via Thread, Bluetooth, and/or Wi-Fi, to name a few examples. Processor or processors 102 may integrate the signals from multiple electrodes to generate a comprehensive EKG reading.

### Data Analysis and Display

The application may perform basic and advanced calculations on the EKG data, such as measuring PR intervals and detecting arrhythmias. The processed data may be displayed in real-time on the device's display 108.

### Data Transmission and Storage

The application may transmit the EKG data to other systems, such as EHRs, printers, and centralized hubs using wireless communication. The data may also be stored locally on the device 100 for future reference or further analysis.

### Additional Features

The system may support “selfie EKG” mode, where users may perform an EKG on themselves using the front-facing camera.

It may operate in various environments, including home, office, and remote locations, providing flexibility for continuous monitoring, e.g., using links such as cellular networks (e.g., T-Mobile; Verizon) and through them or directly to satellite communications systems such as StarLink and other such systems discussed herein.

In an example embodiment, device 100 may use camera 110 to detect the presence and position of the wireless electrodes. The device may read and log the unique identifier signal from each electrode. The device may then display the optimal electrode placement locations on the device's screen (display 108), guiding the user on where to place each electrode accurately.

In an example embodiment, device 100 may verify their positions using cameras 110 once the electrodes are placed. The device may scan the patient's thoracic anatomy to ensure the electrodes are in the correct positions. If any electrodes are incorrectly placed or missing, the device may provide feedback on the display 108, indicating which electrodes need to be adjusted.

In an example embodiment, wireless communication module 106 may receive signals from the electrodes. These signals may be transmitted via communication protocols such as Thread, Bluetooth, and/or Wi-Fi to the device 100. The device may integrate these voltage measurements into an electrocardiogram (EKG) reading.

In an example embodiment, processors 102 may handle the data processing tasks required for analyzing the signals received from the wireless electrodes. The application running on device 100 may perform both basic and advanced calculations on the EKG data, such as measuring PR intervals and detecting arrhythmias. The processed data may then be displayed in real time on the device's display 108.

In an example embodiment, the mobile device may provide real-time feedback on the quality of the EKG signal based on the detected placement of the electrodes. This feedback may include indications of poor signal quality and suggestions for repositioning the electrodes.

In an example embodiment, EKG data may be transmitted from the mobile device to external systems such as EHRs, printers, and hubs using wireless communication protocols. This may involve encrypting the data for secure transmission and ensuring compatibility with various external systems.

In an example embodiment, the mobile device's front-facing camera may be used to guide and verify electrode placement for a self-administered EKG. This may involve capturing images of the patient's chest, analyzing the images to determine electrode positions, and providing visual feedback to the user.

In an example embodiment, the EKG data may be stored locally on the mobile device for future reference or further analysis. This may involve saving the data in a secure format and organizing it for easy retrieval and review.

In an example embodiment, the mobile device may adjust the display to fit different screen dimensions and aspect ratios for optimal viewing of EKG data. This may involve scaling the display elements and reorganizing the layout to ensure the data is clearly visible on various screen sizes.

In an example embodiment, the mobile device may calculate heart rate variability (HRV) from the EKG data. This may involve analyzing the time intervals between heartbeats and providing metrics that indicate the variability in heart rate.

In an example embodiment, the mobile device application may provide notifications for irregular heart rhythms detected during the EKG monitoring. This may involve continuously analyzing the EKG data and alerting the user if any abnormal rhythms are detected.

In an example embodiment, the mobile device may enable sharing of EKG data securely with healthcare providers through encrypted channels. This may involve encrypting the data, establishing a secure connection, and transmitting the data to authorized recipients.

In an example embodiment, the EKG data may be integrated with fitness tracking applications on the mobile device, e.g., Apple Health, Zones, HeartWatch, and/or any other current or future fitness tracking applications on the mobile device. This may involve synchronizing the EKG data with other health metrics and providing a comprehensive view of the user's health.

In an example embodiment, the mobile device may record patient symptoms and correlate them with the EKG data. This may involve allowing the user to input symptoms, timestamping the inputs, and linking them to the corresponding EKG data.

In an example embodiment, the mobile device may perform noise reduction on the received EKG signals to improve data accuracy. This may involve filtering out interference and artifacts from the signal to produce a clearer EKG reading.

In an example embodiment, the mobile device may use augmented reality (AR) to assist in electrode placement. This may involve overlaying visual cues on the real-world image of the patient's chest to guide the placement of electrodes.

In an example embodiment, the mobile device may generate reports summarizing the EKG findings for patient records. This may involve compiling the analyzed data into a structured report format and making it available for printing or electronic sharing.

In an example embodiment, the mobile device may be configured to support multiple languages for user instructions and feedback. This may involve providing language options in the application settings and translating all interface elements and notifications.

In an example embodiment, the mobile device may use machine learning algorithms to improve the accuracy of EKG data interpretation over time. This may involve training algorithms on collected data to enhance their ability to recognize patterns and detect abnormalities accurately.

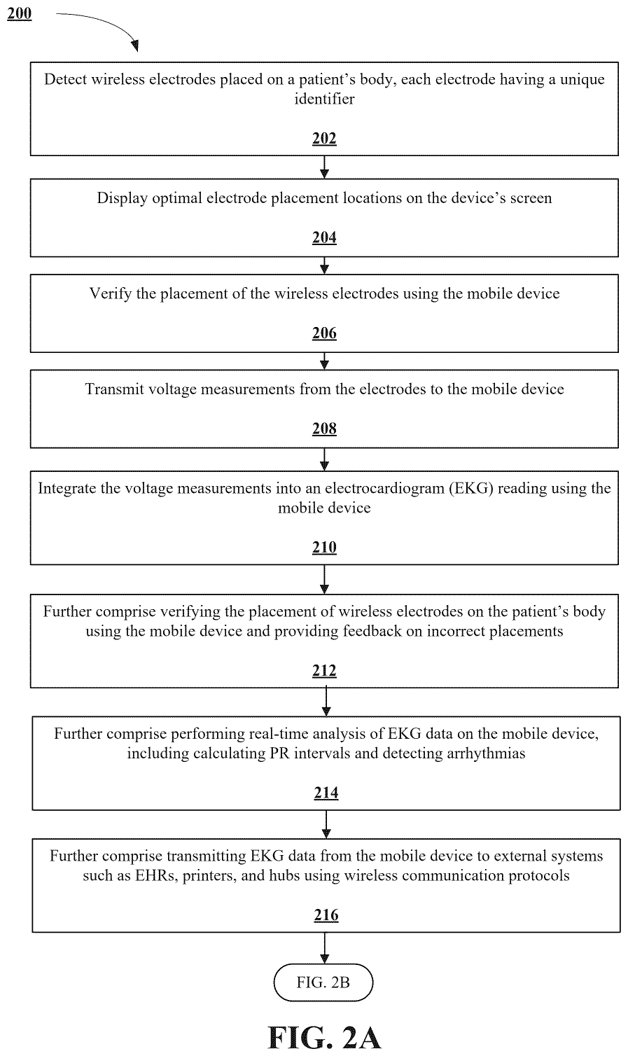

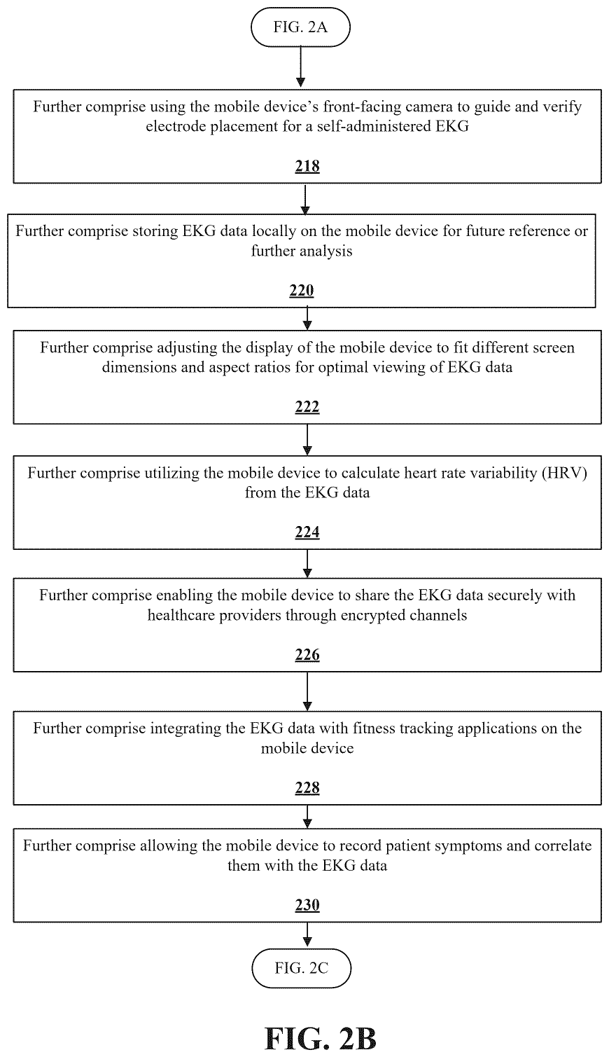

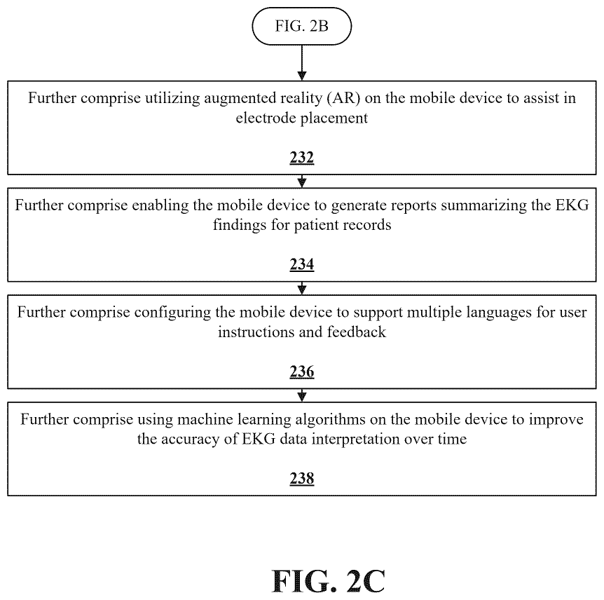

FIGS. 2A-2C illustrates a flowchart 200 outlining the method for wireless EKG monitoring. The process begins with detecting wireless electrodes placed on a patient's body (step 202), each electrode having a unique identifier. The system displays optimal electrode placement locations on the device's screen (step 204). The placement of the wireless electrodes may then be verified using the mobile device (step 206). The system transmits voltage measurements from the electrodes to the mobile device (step 208). The system may integrate these measurements into an electrocardiogram (EKG) reading using the mobile device (step 210). In some embodiments, an example system may also be able to analyze an image (and/or video) at the patient's chest and show where the electrodes should be placed. Then after placement the example system may determine if the positions are correct and if not may show how to move them to a correct place, e.g., on the patient's chest. This point may be used for the training features of the system. For example, these features may allow untrained personnel to accurately place the electrodes and help train them to do so in the future.

Detecting wireless electrodes (step 202) involves scanning the patient's body using cameras on the device to identify the presence of electrodes. The unique identifier signals from each electrode are read and logged by the device, allowing it to recognize the placement configuration.

Displaying optimal electrode placement locations (step 204) includes analyzing the patient's thoracic anatomy using the mobile device's cameras. The system compares the detected electrode positions with pre-defined optimal placement patterns and displays visual guidance on the device's screen indicating where to place or adjust the electrodes.

Verifying the placement of the wireless electrodes (step 206) involves scanning the electrode positions using the device's cameras to ensure correct placement. The device provides real-time feedback on the screen about the correctness of electrode placement, indicating any incorrectly placed or missing electrodes and suggesting adjustments.

Transmitting voltage measurements (step 208) includes capturing voltage signals from each electrode and transmitting the captured signals to the mobile device via communication protocols such as Thread, Bluetooth, and/or Wi-Fi. This ensures secure and accurate transmission of the data to the device for further processing.

Integrating voltage measurements into an EKG reading (step 210) involves processing the received voltage signals using the device's processors. The system performs calculations to convert the voltage signals into EKG data, including PR intervals and arrhythmia detection, and displays the processed EKG reading in real-time on the device's screen for immediate analysis and review. It will also be understood that, in some example embodiments, the raw data may, in some example embodiments, be stored for possible transmission to another system (e.g., the cloud, etc.) for additional processing.

The method may further comprise providing real-time feedback on the quality of the EKG signal based on the detected placement of the electrodes (step 212). This includes analyzing the signal strength and consistency to ensure accurate data collection and suggesting adjustments if necessary. Additionally, verifying the placement of wireless electrodes on the patient's body using the mobile device may provide feedback on incorrect placements (step 214). This involves scanning the electrodes, identifying any discrepancies in placement, and displaying corrective guidance.

Performing real-time analysis of EKG data on the mobile device (step 216) may include calculating PR intervals and detecting arrhythmias, ensuring immediate and accurate assessment of the heart's electrical activity. The method may also comprise transmitting EKG data from the mobile device to external systems such as EHRs, printers, and hubs using wireless communication protocols (step 218). This involves securely sending the data for storage, printing, or further analysis. For example, as discussed above, the raw data may, in some example embodiments, be stored for possible transmission to another system (e.g., the cloud, etc.) for additional processing.

Using the mobile device's front-facing camera to guide and verify electrode placement for a self-administered EKG (step 220) involves capturing images of the patient's chest, analyzing the images to determine electrode positions, and providing visual feedback. The method may further include selecting the mobile device from an iPad, iPhone, and VisionPro (step 222), ensuring compatibility with various Apple devices.

The wireless electrodes may communicate with the mobile device using a communication protocol comprising at least one of Thread, Bluetooth, and Wi-Fi (step 224), ensuring reliable and versatile data transmission. The method may also comprise storing EKG data locally on the mobile device for future reference or further analysis (step 226), involving saving the data in a secure format and organizing it for easy retrieval.

Adjusting the display of the mobile device to fit different screen dimensions and aspect ratios for optimal viewing of EKG data (step 228) includes scaling and reorganizing display elements. Additionally, utilizing the mobile device to calculate heart rate variability (HRV) from the EKG data (step 230) involves analyzing the time intervals between heartbeats to provide metrics on heart rate variability.

The mobile device application may provide notifications for irregular heart rhythms detected during the EKG monitoring (step 232), continuously analyzing the EKG data and alerting the user if any abnormal rhythms are detected. The method may further comprise enabling the mobile device to share the EKG data securely with healthcare providers through encrypted channels (step 234), involving encrypting the data, establishing a secure connection, and transmitting the data to authorized recipients.

Integrating the EKG data with fitness tracking applications on the mobile device (step 236) may involve synchronizing the EKG data with other health metrics for a comprehensive view of the user's health. The method may also allow the mobile device to record patient symptoms and correlate them with the EKG data (step 238), involving inputting symptoms, timestamping the inputs, and linking them to the corresponding EKG data.

Performing noise reduction on the received EKG signals to improve data accuracy (step 240) involves filtering out interference and artifacts from the signal. Utilizing augmented reality (AR) on the mobile device to assist in electrode placement (step 242) may involve overlaying visual cues on the real-world image of the patient's chest.

Generating reports summarizing the EKG findings for patient records (step 244) involves compiling the analyzed data into a structured report format and making it available for printing or electronic sharing. Configuring the mobile device to support multiple languages for user instructions and feedback (step 246) may involve providing language options and translating interface elements and notifications.

Finally, using machine learning algorithms on the mobile device to improve the accuracy of EKG data interpretation over time (step 248) involves training algorithms on collected data to enhance their pattern recognition and anomaly detection capabilities.

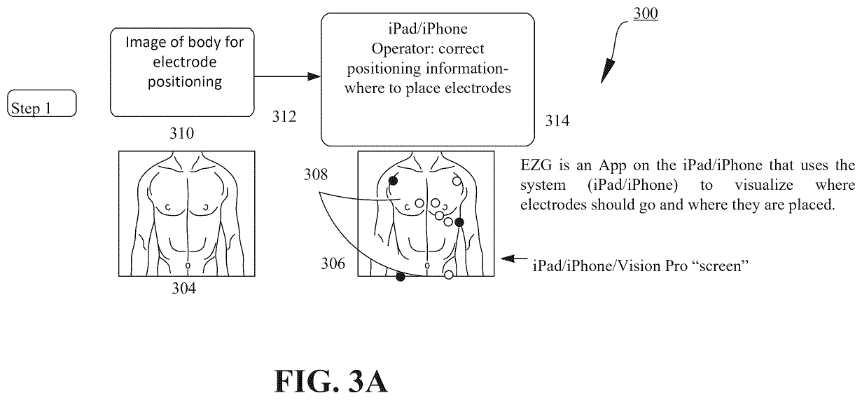

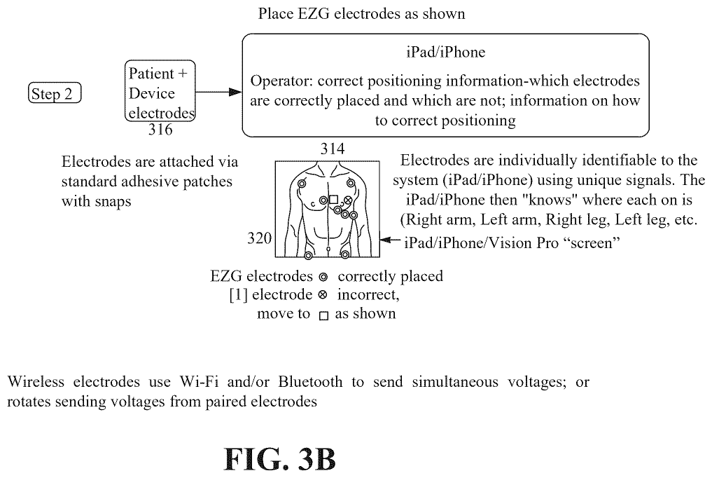

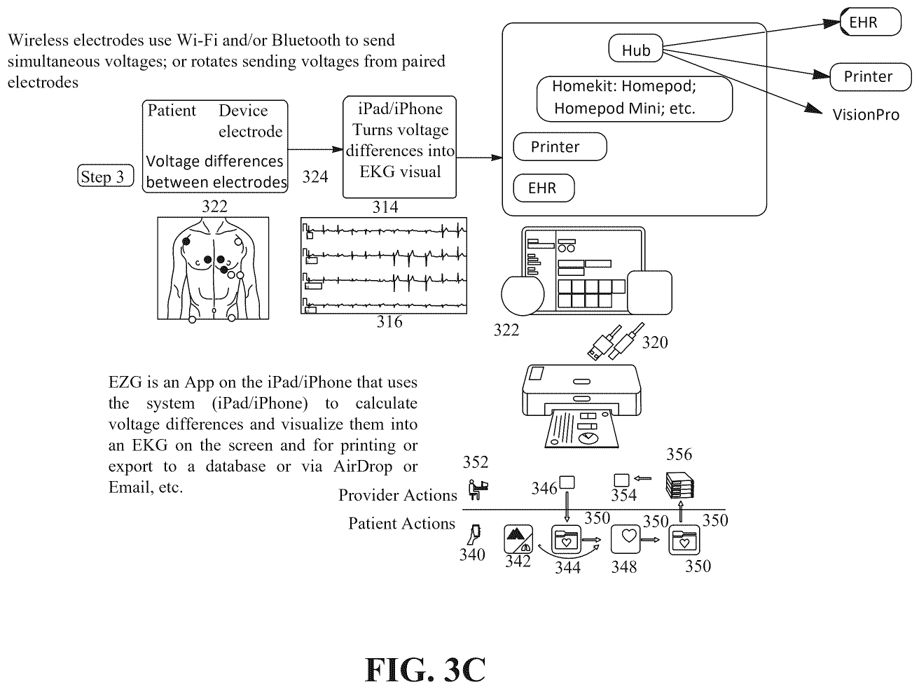

FIGS. 3A-3C presents a detailed data flow diagram 300 illustrating the EZG (example software implementing the EKG). In a first stage (Step 1), imaging device (e.g., ultrasound scanner, optical imaging device, or other body-imaging system) may be used to capture anatomical information of the patient, e.g., an image 304. This imaging 306 may be used to identify the optimal electrode locations 308 on the patient's body. The imaging data 310 may be transmitted via communication link 312 to an electronic device 314, such as an iPad, iPhone, VisionPro, or any other suitable electronic communication device.

The electronic device 314 may host an EZG application that may process the incoming imaging data 310 and may overlay electrode position markers at optimal electrode locations 308 on the patient's body, e.g., onto a body model displayed on graphical user interface (GUI). These markers may guide the user to place electrodes in the correct anatomical locations.

In certain cases, patients may have dextrocardia (i.e., cardiac orientation to the right). The EZG application may be pre-programmed with electrode distribution rules or may analyze imaging data to detect dextrocardia and automatically suggest alternate electrode placement patterns. Thus, system logic may support standard and atypical cardiac orientations.

In the second stage (Step 2), electrodes may be physically attached 316 to the patient at the locations indicated by GUI markers 308. The electrodes may be adhesive patches with snaps or connectors. Each electrode may include an identifier element (e.g., RFID, unique signal, or code) allowing the EZG application to recognize and track electrode identity and placement.

The electronic device may determine electrode placement using a detection module. In some embodiments, the detection module may be implemented as software stored in memory 104 and executed by processors 102 of device 100 (FIG. 1). The detection module may be configured to receive imaging data from cameras 110 and electrode-identification data via wireless communication module 106 to determine electrode presence and position. In an example embodiment, an iPad/iPhone 314 (or other electronic device) may be used to identify correct positioning information for the electrodes. The status of the placement may be indicated on a body diagram 320.

The detection module may identify whether electrodes are on the right arm (RA), left arm (LA), right leg (RL), left leg (LL), or precordial chest positions (V1-V6). Placement verification logic may compare actual versus expected positions and generates feedback indicators (visual alerts on GUI or textual prompts). If misplacement is detected, corrective guidance is presented to the user to reposition electrodes accurately. As noted, detection module and feedback indicators may provide visual/textual guidance to correct misplaced electrodes, ensuring accurate positioning for optimal readings.

In the third stage (Step 3), voltage differences 322 may be measured between electrodes. These analog signals may be converted into digital data streams 324 and transmitted to EZG application, e.g., on the electronic device 314. A processing engine may transform the voltage differences into EKG waveforms 316, which may rendered on a GUI in real time.

The EKG visuals (EKG waveforms 316) may be printed via printer interface 320 exported to database/storage 322, or shared externally using communication module 372 (e.g., AirDrop, email, secure cloud transfer). This enables seamless archiving, analysis, and distribution of patient EKG data. The resulting EKG visuals may be printed. This may enable seamless archiving. This ensures versatile handling of EKG information for clinical or personal use.

In various embodiments, electrodes may be wired or wireless, with wireless modules transmitting signals to electronic device either directly or through a hub. Wireless communication can employ Bluetooth, Thread, Zigbee, Z-Wave, LoRa, HomeKit, Matter, cellular, or satellite connectivity (e.g., Starlink, Viasat), or other current or future protocols used in medical, IoT, or consumer electronics environment.

In some embodiments, a patient (see patient's hand holding electronic device at 340) may download and enroll in a mobile health application (mHealth app) 342 (e.g., an asthma or cardiac monitoring app), which generates patient-generated health data (PGHD). The patient may also download and activate an electronic health record (EHR) patient portal application 344 (e.g., MyChart), which may provide a conduit to the patient's EHR system 346. The patient may then enable operating-system-level sharing of health data through a health aggregation framework 348 (e.g., a mobile device's health app), thereby authorizing exchange of PGHD between the mHealth app 342 and the EHR portal app 344. Once enabled, data 350 from the mHealth app 342 may be transmitted through the health aggregation framework 348 to the EHR portal app 344 and onward to the EHR system 346.

On the provider side, a healthcare provider 352 may initiate an order through the EHR system 346 specifying the type of PGHD requested, as well as collection frequency and thresholds for generating alerts. The provider 352 may then review submitted PGHD 354 within the EHR system 346, which can automatically issue notifications 354 when values exceed configured thresholds or when abnormal data is detected. Periodic summaries and alert notifications 356 may thus be provided to the provider, ensuring seamless integration of patient-generated data into existing clinical workflows and enabling timely review and intervention.

In an aspect, wireless devices may receive input from patch electrodes (voltage changes) and transmit the information to an iPad, iPhone, VisionPro, central hub, or EHR.

In an aspect, the iPad/iPhone/VisionPro application may use the device to detect the patient and show where the patches and devices should go.

In an aspect, the iPad/iPhone/VisionPro application may determine if the patches/patch devices are in the correct places and indicate incorrect placements.

In an aspect, the iPad/iPhone/VisionPro may identify which devices are which by position (right arm, right leg, left arm, left leg, and precordial positions).

In an aspect, the iPad/iPhone/VisionPro may use Thread or similar technology to communicate simultaneously with all devices or rotate through combinations of electrodes.

In an aspect, electrodes may communicate directly with the Apple device or through a hub.

In an aspect, the iPad/iPhone/VisionPro application may integrate signals from pairs or groups of devices to generate an EKG.

In an aspect, the iPad/iPhone/VisionPro application may communicate the EKG to other systems, either directly or through a hub.

In an aspect, the iPad/iPhone/VisionPro application may analyze the EKG for abnormal findings and/or pass the EKG to another system for further analysis.

In an aspect, the system may be applicable to other applications such as an EEG.

In an aspect, the system may support “selfie EKG” mode for user-conducted EKGs.

In an aspect, the system may communicate data through HomeKit devices over distances.

In an aspect, the system may be extendable to other devices via Matter or similar protocols.

In an aspect, the electrodes may be interchangeable, not mandated to one position.

In an aspect, the electrodes may be easy to clean and maintain (possibly sterilizable).

In an aspect, the electrodes may be inexpensive and easily replaced.

In an aspect, the number of electrodes may not be limited to 10.

In an aspect, there may be a possibility of an esophageal electrode.

In an aspect, the system may be used as an ambulatory EKG or Holter monitor replacement.

In an aspect, the system may have applications in military, sports, and preventative care.

In an aspect, the software may allow for electrode selection for atypical cases, e.g., patients with dextrocardia, as discussed above, or other atypical cases.

In an aspect, the software may allow for selecting a subset of electrodes for specific cases.

In an aspect, the system may support using Apple technology to function without a network.

In an aspect, the system may include a charging tray for electrodes.

In an aspect, the electrodes may have solar charging for remote environments.

In an aspect, the system may have a satellite connection, such as, for example, a StarLink satellite connection, Viasat, HughesNet, OneWeb, Amazon Project Kuiper, Telesat, AST SpaceMobile, Iridium, or any other current or future satellite connection, e.g., for remote environments.

In an aspect, the system may be mobile and connect through cellular networks for continuous monitoring.

In an aspect, the EZG Application may adjust to any current (or future) Apple device (or other manufacturer's devices) screen dimensions and aspect ratio.

In an aspect, the system may provide feedback on incorrect placements of the wireless electrodes and suggest corrective actions.

In some examples, a typically correct placement may be flagged as incorrect, e.g., for atypical cases such as patients with dextrocardia. In such an instance the system may also provide feedback on incorrect placements of the wireless electrodes and suggests corrective actions.

Wireless Multi-Node ECG Architecture Without Earth Ground. In some embodiments, a fully wireless electrocardiogram (ECG/EKG) architecture is provided that eliminates the need for a tethered return or earth ground while preserving diagnostic common-mode rejection and inter-lead timing. The system includes a plurality of adhesive measuring nodes each configured to acquire a local skin potential relative to an internal reference, a wireless common-mode suppression/drive (CMS/DRL) node placed at a right-leg location, and an aggregation device (e.g., a mobile device or hub). Each node is battery powered and communicates wirelessly with the aggregation device.

Single-Contact Measuring Nodes. In one embodiment, each measuring node (e.g., RA, LA, LL, V1-V6) comprises: (i) a biopotential analog front-end (AFE) with a programmable gain amplifier referenced to an internal mid-supply node (virtual reference), (ii) an input protection network including an electrostatic-discharge element, a series resistor, and a small shunt capacitor to attenuate radio-frequency interference, (iii) a radio/MCU subsystem configured to packetize and transmit timestamped samples, and (iv) a rechargeable battery and power management circuitry. The measuring input may be a single electrode contact to the skin; no wired ground or return is required at the node.

Wireless Common-Mode Suppression Without Earth Ground. In certain embodiments, a dedicated CMS/DRL node is positioned at a right-leg location and includes a common-mode sensor input and a drive output. A drive amplifier inverts the sensed common-mode and injects a small current into the body through a high-value series resistor (e.g., ≥0.5 MΩ, preferably ≥1 MΩ) to collapse mains and motion-induced common-mode prior to the measuring AFEs. A feedback integrator (e.g., 0.5-10 Hz loop bandwidth) may be included to ensure loop stability. The CMS/DRL node operates without any wired connection to earth ground, maintaining patient isolation while preventing AFE saturation.

Time Alignment and Resampling. To preserve diagnostic inter-lead phase relationships, each node may attach a high-resolution timestamp to each sample using a crystal-derived timebase. The aggregation device estimates per-node clock offset and drift and resamples incoming streams onto a common temporal grid (e.g., 250-500 samples per second) prior to lead derivation. In some embodiments, inter-lead alignment better than about 0.5-2.0 ms is achieved.

Virtual Reference and 12-Lead Reconstruction. The aggregation device may compute a virtual reference, such as a Wilson Central Terminal (WCT), from limb potentials (e.g., WCT=(RA+LA+LL)/3) and derive standard diagnostic leads. In one example, limb leads are computed as I=LA−RA, II=LL−RA, and III=LL−LA; augmented leads as aVR=RA−(LA+LL)/2, aVL=LA−(RA+LL)/2, and aVF=LL−(RA+LA)/2; and precordial leads as Vk=Vk(raw)−WCT for k ∈{1 . . . 6}. By combining virtual reference computation with precise time alignment, accurate 12-lead electrocardiograms are reconstructed from fully wireless, single-contact measuring nodes.

Diagnostic Filtering and Quality Assurance. The aggregation device may apply diagnostic-profile filtering, for example a 0.05-150 Hz band-pass with an optional 50/60 Hz notch and baseline-wander control, to maintain fidelity suitable for clinical interpretation. Quality-assurance logic can continuously evaluate signal plausibility, such as checking Einthoven's identity (I+III≈II) against a threshold, monitoring AFE headroom to detect saturation, and performing lead-off detection. In some embodiments, acquisition and/or display can be gated until quality metrics satisfy configured thresholds.

Safety and Isolation. All body-worn nodes may be battery-powered and isolated from earth ground. The CMS/DRL injection path may include current limiting via a high-value series resistor and protective components at the drive electrode. The input networks of measuring nodes may employ low-leakage components and mechanical features (e.g., guarded traces) to maintain high input impedance and minimize artifact susceptibility. The system may be configured to meet applicable medical electrical safety and performance standards depending on the intended use.

Representative Parameter Ranges. Without limitation, representative values include: sampling rate of approximately 250-500 samples per second per channel; programmable gain amplifier gain between about 1 and 12; series input resistor between about 500 Ω and 5 kΩ; shunt capacitor between about 1 nF and 10 nF; CMS/DRL series resistor of at least about 0.5 MΩ, preferably at least about 1 MΩ; and CMS/DRL loop bandwidth between about 0.5 Hz and 10 Hz. In some embodiments, inter-lead timing skew after resampling is maintained at or below about 0.5-2.0 ms.

Wireless Transport and Throughput. In one example, Bluetooth® Low Energy (BLE) is used between nodes and the aggregation device; other protocols (e.g., Zigbee®, Wi-Fi®, UWB, Thread®, or body-area communications) may be used in alternative embodiments. Timestamped batching and lightweight compression (e.g., delta or predictive coding) may be employed to maintain aggregate throughput for ten nodes at diagnostic sampling rates.

Technical Effects and Advantages. The disclosed architecture improves patient comfort and mobility by eliminating tethered leads while maintaining diagnostic-quality signals. Active common-mode suppression at the body reduces interference and prevents AFE saturation even in high-noise environments, and time-aligned digital reconstruction preserves inter-lead phase accuracy needed for ST-segment and vector analysis. Continuous software-based quality checks further enhance reliability in clinical and home settings.

Integration With Placement Guidance. The foregoing embodiments are compatible with camera-assisted placement and verification features described elsewhere herein. For example, camera-based thoracic anatomy recognition and augmented-reality overlays may guide placement of both measuring nodes and the CMS/DRL node; after placement, the system may combine optical verification with electrical quality-assurance metrics to confirm correct configuration.

Exemplary Components (Non-Limiting). In certain embodiments, the AFE can be a delta-sigma ECG front-end with internal reference and DRL amplifier; the radio can be a BLE 5.x system-on-chip; protective elements can include an ESD diode at the electrode pad; and power management may include a rechargeable cell with over-current/over-temperature protection. Component values and selections are illustrative and not limiting.

Alternative Embodiments. In some embodiments, a body-worn hub may aggregate node streams and forward a consolidated signal to a mobile device. Clock synchronization can be enhanced using Bluetooth isochronous channels, a periodic timing beacon, or a pilot-tone alignment method. In other embodiments, a legacy wired reference may be optionally provided for compatibility modes while still supporting fully wireless operation.

Use Cases. The system may operate in diagnostic 12-lead mode (e.g., 0.05-150 Hz, ˜500 samples per second) or monitoring/ambulatory modes with adjusted bandwidth and sampling. Applications include emergency care, clinical diagnostics, ambulatory Holter replacement, home monitoring, athletic/rehabilitation monitoring, and military or remote deployments.

The preceding disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it is understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like) and may be used interchangeably with “one or more.” The phrase “only one” or similar language is used where only one item is intended. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

One or more elements or aspects or steps, or any portion(s) thereof, from one or more of any of the systems and methods described herein, may be combined with one or more elements or aspects or steps, or any portion(s) thereof, from one or more of any of the other systems and methods described herein and combinations thereof, to form one or more additional implementations and/or claims of the present disclosure.

One or more components, steps, features, and/or functions illustrated in the figures may be rearranged and/or combined into a single component, block, feature, or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification do not necessarily refer to the same embodiment.

Some portions of the detailed description are presented in terms of algorithms and symbolic representations of operations on data bits within a computer memory. These algorithmic descriptions and representations are the methods used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared or otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following disclosure, it is appreciated that throughout the disclosure terms such as “processing,” “computing,” “calculating,” “determining,” “displaying” or the like, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. It will be appreciated that a variety of programming languages may be used to implement the teachings of the invention as described herein.

The figures and the description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this Application. As will be understood by those familiar with the art, the present invention may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

Furthermore, as will be apparent to one of ordinary skill in the relevant art, the modules, routines, features, attributes, methodologies and other aspects of the present invention can be implemented as software, hardware, firmware or any combination of the three. Also, wherever a component, an example of which is a module, of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming.

Additionally, the present invention is in no way limited to implementation in any specific programming language, or for any specific operating system or environment. Accordingly, the disclosure of the present invention is intended to be illustrative, but not limiting, of the scope of the present invention, which is set forth in the following claims.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

What is claimed is:

1. A method for wireless electrocardiogram (EKG) monitoring, comprising:
detecting wireless electrodes placed on a patient's body, each electrode having a unique identifier;
displaying optimal electrode placement locations on the device's screen;
verifying the placement of the wireless electrodes using the mobile device;
transmitting voltage measurements from the electrodes to the mobile device; and
integrating the voltage measurements into an EKG reading using the mobile device.

2. The method of claim 1, wherein the mobile device provides real-time feedback on quality of the EKG signal based on detected placement of the electrodes.

3. The method of claim 2, wherein the mobile device provides feedback on incorrect placements of the wireless electrodes and suggests corrective actions.

4. The method of claim 1, further comprising performing real-time analysis of EKG data on the mobile device, including calculating PR intervals and detecting arrhythmias.

5. The method of claim 1, further comprising transmitting EKG data from the mobile device to external systems such as EHRs, printers, and hubs using wireless communication protocols.

6. The method of claim 1, further comprising using the mobile device's front-facing camera to guide and verify electrode placement for a self-administered EKG.

7. The method of claim 1, wherein the mobile device comprises one of an iPad, an iPhone, and a VisionPro.

8. The method of claim 1, wherein the wireless electrodes communicate with the mobile device using a communication protocol comprising at least one of Thread, Bluetooth, and Wi-Fi.

9. The method of claim 1, further comprising storing EKG data locally on the mobile device for future reference or further analysis.

10. The method of claim 1, further comprising adjusting the display of the mobile device to fit different screen dimensions and aspect ratios for optimal viewing of EKG data.

11. The method of claim 1, further comprising utilizing the mobile device to calculate heart rate variability (HRV) from the EKG data.

12. The method of claim 1, wherein the mobile device application provides notifications for irregular heart rhythms detected during the EKG monitoring.

13. The method of claim 1, further comprising enabling the mobile device to share the EKG data securely with healthcare providers through encrypted channels.

14. The method of claim 1, further comprising integrating the EKG data with fitness tracking applications on the mobile device.

15. The method of claim 1, further comprising allowing the mobile device to record patient symptoms and correlate them with the EKG data.

16. The method of claim 1, wherein the mobile device performs noise reduction on the received EKG signals to improve data accuracy.

17. The method of claim 1, further comprising utilizing augmented reality (AR) on the mobile device to assist in electrode placement.

18. The method of claim 1, further comprising enabling the mobile device to generate reports summarizing the EKG findings for patient records.

19. The method of claim 1, further comprising configuring the mobile device to support multiple languages for user instructions and feedback.

20. The method of claim 1, further comprising using machine learning algorithms on the mobile device to improve the accuracy of EKG data interpretation over time.
